(19) United States
(12) Patent Application Publication
(10) Pub. No.: US 2005/0245956 A1
(43) Pub. Date: Nov. 3, 2005

(54) Insertion device for needle units

(76) Inventors: Urs Steinemann, Arch (CH); Martin Wyss, Konolfingen (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/137,715

(22) Filed: May 25, 2005

Related U.S. Application Data
(63) Continuation of application No. PCT/EP03/12237, filed on Nov. 3, 2003.

(30) Foreign Application Priority Data
Nov. 26, 2002 (DE) 102 55 133.2

(51) Int. Cl. A61M 3/00; A61B 17/34; A61B 10/00

(52) U.S. Cl. 606/185; 604/44

(57) ABSTRACT

A device for at least partially inserting a needle unit into a tissue, the device including a housing, a support with a receiving element and an actuation device, wherein the needle unit is detachably received in the receiving element and the support can be displaced relative to the housing from a first position to a second position in which the needle unit is at least partially inserted in the tissue, and wherein the support is mounted inside the housing so that it can be moved about an axis.

### CROSS-REFERENCE TO RELATED APPLICATION(S)

This application is a continuation of International Patent Application No. PCT/EP2003/012237, filed on Nov. 3, 2003, which claims priority to German Application No. 102 55 133.2, filed on Nov. 26, 2002, the contents of which are incorporated by reference in their entirety.

### BACKGROUND

The present invention relates to devices and methods for inserting a needle at least partially into tissue and, more particularly, to a device and method for inserting a needle unit or needle head of an infusion device into human tissue.

Many areas of medicine necessitate percutaneous administration of a liquid product or withdrawal of a test medium from body tissue. For this purpose, it is customary to apply a needle unit to the surface of a patient's tissue, the needle of said needle unit penetrating into the tissue. The needle in this case can be made of metal or it can also be formed by a flexible cannula. A suitable administration device or withdrawal device can be attached to the needle unit placed on the tissue. Such a device can, for example, be an infusion pump for insulin. In many treatment methods, repeated administration of the product is required, or, in the case of continuous administration, it is necessary to change the needle unit, entailing frequent insertion of a needle unit into the tissue. The patient should therefore be able to apply a needle unit to the tissue himself. An insertion device for the needle unit may help the patient perform this procedure. It may help him overcome his reluctance to insert a needle into his own body tissue. Moreover, an insertion device can ensure that the needle unit is arranged in a correct position on the tissue and that the needle is introduced in a desired position into the tissue.

WO 99/33504 describes an insertion device for inserting a needle of a needle unit through a patient's skin. The insertion device has a cylindrical housing in which a support for the needle unit is accommodated in such a way as to be displaceable along the longitudinal axis of the cylinder housing. With the aid of the support, the needle unit can be moved from a retracted position, inside the housing, along the axis of the housing to an extended position in which the needle of the needle unit protrudes from the housing. The support can be moved from the retracted position to the extended position with controlled force and speed by means of a drive device. To trigger the drive device, the upper end of the housing directed away from the tissue is provided with a press button which, when pressed into the housing, moves the support from the retracted position to the extended position. In the extended position, the needle unit is released from the support and remains on the patient's tissue.

WO 98/33549 discloses a similar insertion device with a cylindrical housing in which a piston-like support for the needle unit is arranged inside the cylinder housing and is held in a prestressed retracted position by a spring and by a securing means. When the securing means is released, the support is moved with a predetermined force and speed along the axis of the cylinder housing to an extended position toward the patient's tissue, as a result of which the needle of the needle unit is inserted into the tissue.

### SUMMARY

It is an object of the present invention to make available an insertion device for a needle unit which permits a preferred and substantially exact or precise orientation of the needle unit upon insertion and which is easy to use.

The object is addressed by a device for inserting a needle unit in accordance with the present invention, the device comprising, in one embodiment, a housing, a support with a receiving element which receives the needle unit in a releasable manner, said support being movable relative to the housing, and an actuation element for triggering the movement of the support, wherein the support is mounted pivotably about an axis inside the housing.

To insert a needle unit at least partially into tissue, i.e. to introduce a needle of the needle unit into the tissue, an insertion device according to the invention comprises a housing, which is preferably designed as an elongate body with a bearing surface along the long side of the body for application to the tissue. A support, with a receiving element which receives the needle unit in a releasable manner, is arranged at least partially inside the housing. According to the invention, the support is mounted pivotably about an axis inside the housing, such that it can be pivoted relative to the housing. The support is preferably pivoted from a first position above the tissue to a second position in which the needle unit is inserted at least partially into the tissue. The support preferably comprises at least one lever arm which, at a rear end, is mounted pivotably inside the housing and, at a front end, has the receiving element for the needle unit. The pivoting movement of the lever arm from the first position to the second position is effected by an actuation element.

The insertion device according to the invention can be arranged with the bearing surface on the long side of the housing placed on the tissue, such that a large contact area between tissue and housing is obtained and loss of the insertion position of the needle unit during the insertion movement is substantially avoided. The insertion device is placed on the tissue by arranging the device parallel to the tissue surface. The user does not directly associate this parallel position with a needle insertion procedure in which the needle unit is moved substantially perpendicular with respect to the tissue. It is therefore easy for the user to overcome his disinclination to insert a needle into his skin.

In a preferred embodiment of the insertion device, the actuation element holds a lever arm in the first position in a prestressed state above the tissue. For this purpose, it is possible, for example, to use a torsion spring which is arranged between lever arm and housing. By means of a trigger device, the lever arm can be released from the prestressed state so that it is pivoted from its first position above the tissue to the second position in which the needle of the needle unit protrudes into the tissue. The force and speed with which the lever arm is guided toward the tissue can be determined by the choice of spring with the desired spring power. The trigger device is advantageously arranged on a side of the housing directed away from the bearing surface and can, for example, be formed by a button or a slide which, when pressed or displaced relative to the housing, cancels the prestressing of the lever arm. The second position of the lever arm, in which the needle of the needle unit is inserted into the tissue, is preferably determined by a limit stop on the housing, the lever arm abutting against this limit stop during the pivoting movement after its release from the prestressed state. The limit stop is advantageously arranged in such a way that it arrests the insertion movement at a point in time when the needle of the needle unit has been introduced to a desired depth into the tissue. This therefore avoids the application of excessive pressure on the tissue and, possibly also associated with this, too deep an insertion of the needle.

The support, in some preferred embodiments, at least one lever arm, has a receiving element for a needle unit, which receiving unit orients the needle unit substantially in the direction of pivoting. The receiving element can be formed, for example, by a simple click-type closure into which a needle unit can be releasably clicked. It is also possible to provide the receiving element with a sliding or plug-in connection for holding the needle unit. In principle, any connection between receiving element and needle unit is suitable which holds the needle unit in a desired position during insertion and which is easily releasable after the insertion.

During the pivoting movement of the support, the needle of the needle unit executes a circle movement in which, relative to the tissue, it changes its position in a parallel direction to the tissue surface and changes its rotation angle position with respect to the tissue. After the tip of the needle has been introduced to the desired depth of insertion, i.e. during the insertion movement of the needle inside the tissue, the change of position and of angle may be painful for the patient if the change happens to be too great. It is therefore desirable to compensate the change of position and of angle of the needle unit during the insertion movement.

In a preferred embodiment of an insertion device according to the invention, the support is therefore mounted pivotably in the housing in such a way that the receiving element together with the needle unit maintains its rotation angle position relative to the tissue during the pivoting movement. The angle orientation of the needle of the needle unit, which has been chosen when placing the housing of the insertion device on the tissue, remains unchanged during insertion of the needle unit. For this purpose, the support is provided with at least two lever arms which are arranged offset in the direction of pivoting. At their front end, the lever arms are secured in an articulated manner, and at a distance from one another, on a beam of the receiving element for the needle unit, which beam is oriented in the direction of the tissue. At their rear end, the two lever arms are secured in an articulated manner on the housing and spaced apart one above the other in the direction of pivoting. In this connection, it is advantageous if the distance between the securing points on the beam of the receiving element and the securing points on the housing is substantially equal, so that the two lever arms are arranged in parallel. The geometry of the lever arms, of the beam and of the housing side between the rear securing points then forms a parallelogram, with angles that change during the pivoting movement. The lever arms of a support configured in this way have, at their front end, a receiver rotation point on the joints about which the receiving element for the needle unit turns relative to the lever arm. At their rear end, they have a housing rotation point about which the lever arm turns relative to the housing.

During a pivoting movement of the support from the first position above the tissue to the second position with at least partially inserted needle unit, the parallel lever arms pivot about the receiver rotation point and also about the housing rotation point. In this process, the beam of the receiving element remains in its angle orientation with respect to the tissue, so that the needle unit is lowered in this orientation onto the tissue. In this way it is possible to avoid scratching or tearing open of the tissue by the pivoting movement during insertion of the needle unit.

In another preferred embodiment of the insertion device according to the present invention, the support is provided with a guide device by means of which the support is guided in such a way that the needle unit is guided along a straight line during the pivoting movement. In this way, it is possible to compensate the change of position of the needle unit in the parallel direction to the tissue surface, as occurs in the pivoting movement of the lever arm. For this purpose, the guide device is configured in such a way that, in a support with at least one lever arm, the front end of the lever arm executes a rectilinear movement in the direction of the tissue during the pivoting movement, and preferably substantially perpendicular to the tissue. The guide device is formed by at least one guide rail, in some preferred embodiments, arranged on the housing, and by a pin which engages in the guide rail and is preferably formed on the support. However, it is also possible to form a guide rail on the support and, correspondingly, to provide a pin on the housing.

The guide rail can also be designed as a guide slit. Guide rails and pins can in principle be arranged at any desired positions on the housing and on the support, as long as they permit a rectilinear movement of the needle unit. A pin on the lever arm is preferably provided on a housing rotation point at the rear end of a lever arm. The dimensions of the guide rail can then be kept small. In a preferred embodiment of a guide rail, it extends substantially perpendicular to the direction of insertion of the needle unit. Another possibility is to provide a guide rail which extends parallel to the direction of insertion for a pin arranged on the receiver rotation point at the front end of the lever arm. In this connection, it is also possible to provide this rail with two pins which are arranged, spaced apart from one another, on a beam of the receiving element extending parallel to the direction of insertion. Another possibility is one in which a guide rail for a pin arranged at the rear end of a lever arm has a curved configuration, so that the front end of the lever arm is guided along a straight line toward the tissue during a pivoting movement of the lever arm. For this purpose, the axis of the pivoting movement lies, for example, in a housing rotation point lying in a rear area of the lever arm, but before its outermost end. During the pivoting movement, the part of the lever arm from this housing rotation point to the outermost end of the lever arm is pivoted away from the tissue for lowering of the needle unit. For this pivoting movement, the curved guide rail defines a trajectory through which the lever arm is displaced in parallel relative to the tissue surface. The displacement is such that, during the pivoting movement of the lever arm about the housing rotation point, the front end of the lever arm does not execute a circle movement, but a rectilinear movement.

Various combinations of the guide rails described above, and further embodiments of guide rails, are possible. A combination of guide rails and a support which consists of a plurality of lever arms is also possible. All these combinations, however, must permit an inventive rectilinear movement of a needle unit during the pivoting movement of the support from the first position above the tissue to the second position with an at least partially inserted needle unit. The principles of the present invention and illustrative embodiments are set forth with reference to the drawings. Features and characteristics of the invention which are apparent from the drawing are to be considered as belonging to the invention both in isolation and in combination.

### BRIEF DESCRIPTION OF THE DRAWINGS

FIG. 1 is a perspective view of an insertion device according to the present invention,

FIG. 2 is a view, from underneath, of the insertion device in FIG. 1,

FIG. 3 is a schematic detailed view of a bearing for a support on the housing, with a prestressing spring,

FIG. 4 is a schematic side view of an insertion device in a first position above tissue,

FIG. 5 is a schematic side view of the insertion device from FIG. 4 in a second position with a needle inserted into the tissue,

FIG. 6 is a schematic side view of an insertion device with a support comprising two lever arms, in a first position,

FIG. 7 is a schematic side view of the insertion device according to FIG. 6 in a second position,

FIG. 8 is a schematic side view of an insertion device with a guide device at the rear end of the support, in a first position,

FIG. 9 is a schematic side view of the insertion device according to FIG. 8 in a second position,

FIG. 10 is a schematic side view of an insertion device with a guide device at the front end and rear end of a support, in a first position,

FIG. 11 is a schematic side view of the guide device according to FIG. 10 in a second position,

FIG. 12 is a schematic side view of a further embodiment of an insertion device in accordance with the present invention, with a guide device at the front end and rear end of a support in a first position, and

FIG. 13 is a schematic side view of the insertion device according to FIG. 12 in a second position.

### DETAILED DESCRIPTION OF THE INVENTION

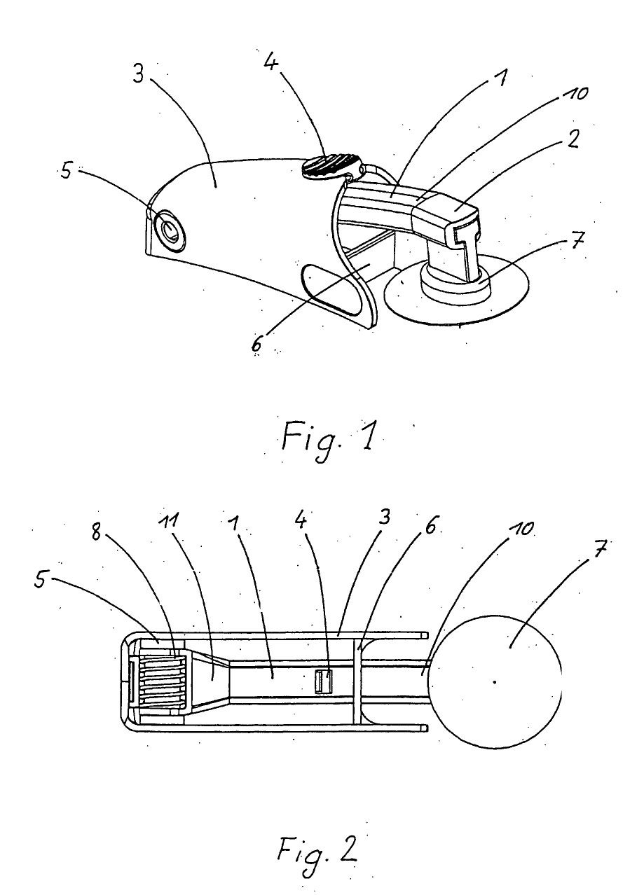

In FIG. 1, an insertion device is shown with a housing 3, a support designed as lever arm 1, and a trigger device 4. At its front end 10 protruding from the housing 3, the lever arm 1 has a receiving element 2 in which a needle unit 7 is received. In principle, it is also possible for the housing 3 to extend over the front end 10 of the lever arm 1, so that the lever arm 1 is completely surrounded by the housing 3. To secure the needle unit 7 in a releasable manner, the receiving element 2 is provided with part of a plug mechanism which is formed by an elongate recess in the form of a slit into which a head part of the needle unit 7 can be pushed, which head part forms another part of the plug mechanism. The rear end 11 of the lever arm 1 is not visible in FIG. 1 and is mounted inside the housing 3 in a rear area via a bearing 5. The bearing 5 forms a pivot axis about which the lever arm 1 can pivot relative to the housing 3.

On its top face, the housing 3 is designed in an elongate curved shape and it has a slightly curved underside via which the insertion device can be placed in a stable manner on tissue. It is also possible to leave the underside of the housing open so that the housing 3 is formed by a kind of curved sleeve open on one side. In a front area, the housing 3 has, on the side placed on the tissue, a limit stop 6 which extends transversely with respect to the pivoting direction of the lever arm. A pivoting movement of the lever arm 1 is checked by this limit stop 6. The trigger device 4 is formed by a slide which, for example by means of a spring, is held in a position in which it secures the lever arm 1. This can be done, for example, by a hook on the slide and by a barb on the lever arm 1. In FIG. 1, the lever arm 1 is shown in a first position in which said lever arm 1 is locked in a position above the tissue by means of the trigger device 4.

In FIG. 2, the insertion device from FIG. 1 is shown from underneath. At the rear end 11 of the lever arm 1, and arranged around the axis of the bearing 5, there is a spring 8 which prestresses the lever arm 1 in the first position above the tissue when locked by the trigger device 4. In the example shown, the spring 8 is represented by a torsion spring. However, it would be conceivable to provide, inside the housing, a tension spring or compression spring between the housing 3 and the lever arm 1, in order to produce the prestressing for the first position. The trigger device 4 engages in a recess on the lever arm 1, which recess forms the barb for the locking.

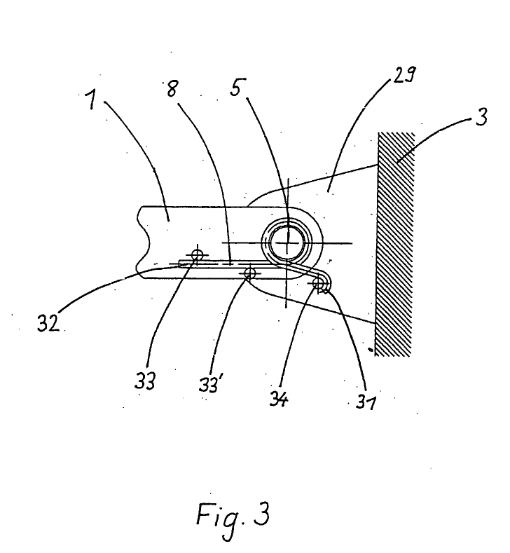

FIG. 3 is a schematic representation of the arrangement of the torsion spring 8 between an extension 29 of a housing side 3 and a lever arm 1 about a bearing 5. One end 32 of the spring 8 is held by projections 33, 33′ arranged on the lever arm 1, and another end 31 of the spring is held by a projection 34 on the housing extension 29. The spring 8 is prestressed between the housing extension 29 and the lever arm 1. By actuating the trigger device 4, the lever arm 1 is released from the locked, prestressed position, so that a pivoting movement of the lever arm 1 about the bearing 5 commences.

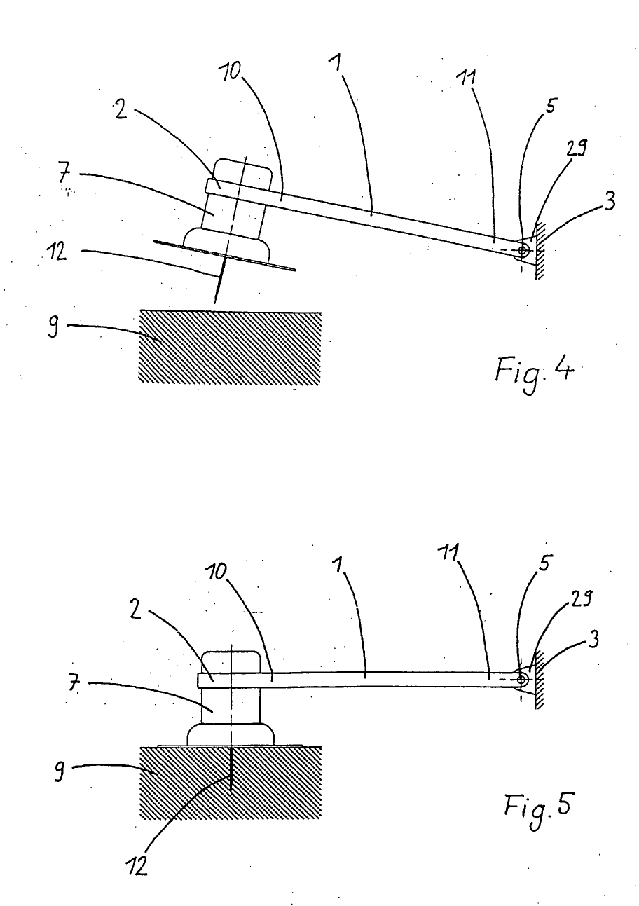

FIGS. 4 through 13 show schematic representations of different embodiments of an insertion device according to the invention. The housing 3 there is indicated only by an inside face of the housing, and the tissue by a tissue surface 9. In these schematic figures, components of the insertion device have been omitted which do not contribute greatly to an understanding of the particular embodiment. In particular, the trigger device 4 and the limit stop 6 are not shown in the schematic figures. It is clear, however, that the embodiments according to FIGS. 4 through 13 are similar in their complete form to the embodiment according to FIG. 1, for example.

FIG. 4 shows a first embodiment of the insertion device in a first position above the tissue surface 9. At a rear end 11, the lever arm 1 is mounted on a bearing arranged on the extension 29 of the housing side 3. At its front end 10, the lever arm 1 has a receiving element 2 in which a needle unit 7 with a needle 12 is received. In the example shown, the needle unit 7 is pushed through an opening in the receiving element 2 and is held releasably by the receiving element 2. The needle 12 of the needle unit 2 is oriented in the direction of the tissue surface 9.

In FIG. 5, the insertion device from FIG. 4 is shown in a second position in which the needle 12 of the needle unit 7 is inserted into the tissue surface 9. To do this, the lever arm 1 has been pivoted about the axis of the bearing 5 relative to the housing side 3 in the direction of the tissue.

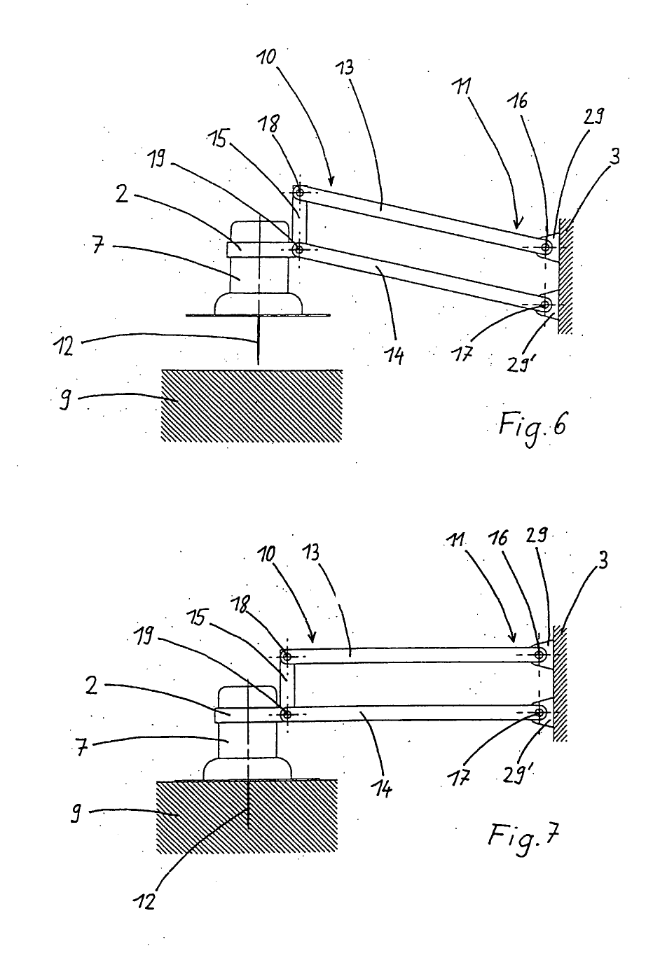

FIG. 6 shows a second embodiment of an insertion device according to the invention in which a support for the needle unit 7 comprises two lever arms 13 and 14 and also a receiving element 2 with a beam 15 extending substantially in the direction of insertion. In the direction of pivoting running through the plane of the drawing, the two lever arms 13 and 14, spaced apart, are mounted alongside one another at their rear end 11 on two bearings 16 and 17 arranged on the housing side 3. The bearings 16 and 17 form housing rotation points of the lever arms 13 and 14. At their front end 10, the two lever arms 13 and 14 are mounted, in an articulated manner, on the beam 15, likewise spaced apart in the direction of pivoting in bearings 18 and 19. The bearings 18 and 19 form receiver rotation points for the lever arms. In this arrangement, a parallelogram is formed between the lever arms 13 and 14, the beam 15 and the housing side 3. In the view shown in FIG. 6, the needle unit 7 is arranged in a first position with the needle 12 at an angle of 90° above the tissue surface 9. However, it is also possible, through the geometry of the housing side 3 and of the receiving element 2, to choose another angle setting with respect to the tissue surface 9.

In FIG. 7, the insertion device from FIG. 6 is shown in a second position in which the needle 12 of the needle unit 7 is inserted into the tissue surface 9. To do this, the lever arms 13 and 14 have been pivoted in parallel about the housing rotation points of the bearings 16 and 17 relative to the housing side 3. At the same time, the beam 15 has been turned about the receiver rotation points in the bearings 18 and 19 relative to the lever arms 13 and 14. By virtue of the geometry of the lever arms 13 and 14, the housing side 3 and the beam 15, the housing side 3 and the beam 15 remain parallel to one another during the pivoting movement. As a result, the needle unit 7 remains in its selected angle position relative to the tissue surface 9 during the pivoting movement. A change in the rotation angle position of the needle 12 relative to the tissue, as described at the outset, is avoided in this embodiment of the insertion device. In the embodiment in FIGS. 6 and 7, it is in principle possible that the bearings 16 and 17 and the bearings 18 and 19 of the respective lever arms are not both arranged in the pivoting plane, as long as an arrangement offset in the pivoting direction is chosen. In this case it is possible, for example, to arrange the bearings 16 and 17 on different inner sides of the housing.

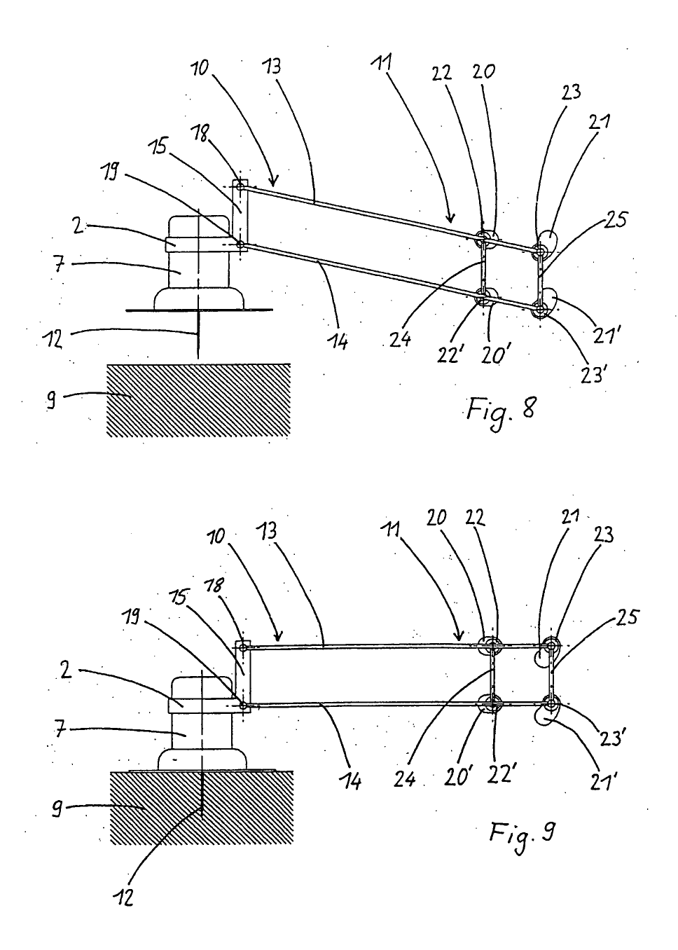

FIG. 8 shows a third embodiment of the insertion device according to the invention in which guide rails 20, 20′ and 21, 21′ of a guide device for the support are shown diagrammatically. In FIG. 8, as also in FIGS. 9 through 13, guide rails are shown as depressions let into an inside surface of the housing, the housing or inside surface having been omitted for the sake of clarity of the figures.

The embodiment of the insertion device according to FIG. 8 has a support with two lever arms 13 and 14 which, at their front end 10, are mounted on a beam of the receiving element 2 in bearings 18 and 19, as in FIG. 6. By means of the receiving element 2, the needle unit 7 is arranged with the needle 12 perpendicularly above the tissue surface 9.

At the rear end 11 of the lever arms, each of said lever arms is provided with a straight guide rail 20, 20′ extending substantially perpendicular to the direction of movement of the needle unit, and, at a distance from these guide rails, there is a curved guide rail 21, 21′, respectively. Arranged on the lever arms 13 and 14 there are cylindrical pins 22, 22′ and 23, 23′ which lie opposite the guide rails 20, 20′ and 21, 21′ and engage displaceably in the guide rails. The width of the guide rails is slightly greater than the diameter of the pins 22, 22′ and 23, 23′, so that these are movable along the guide rails. The pins 22, 22′ belonging to the straight guide rails 20, 20′ form a housing rotation point about which the lever arms are pivoted from the first position to the second position during pivoting of the support.

The pins 22, 22′ are not arranged at the outer end of the lever arms 13 and 14 but instead at a slight distance before this end in relation to the overall length of the lever arms. Secured at the outer end of the lever arms 13 and 14 are the pins 23, 23′ which engage in the guide rails 21, 21′. The guide rails 21, 21′ have a profile which is curved in such a way that, during a pivoting movement of the lever arms 13 and 14, the front end 10 is guided along a straight line in order to permit a rectilinear movement of the needle unit toward the tissue surface 9. For this purpose, the guide rails 21, 21′ are designed in an arc shape from a lower and forward point in the housing to an upper and rearward point in the housing, i.e. the guide rails 21, 21′ extend like arcs around the guide rails 20, 20′. The arc corresponds to a pivoting movement which the rear part of the lever arms executes, measured from the housing rotation point, i.e. the pins 22, 22′, as far as the outer end of the lever arms, i.e. the pins 23, 23′, and which is superposed by the parallel displacement of the lever arms required by the described circle movement of the needle unit during the pivoting movement. The distance, parallel to the tissue surface, between the front and rear points of the guide rails 21, 21′ corresponds to a parallel displacement of the needle unit 7 in a pivoting movement. The distance, perpendicular to the parallel displacement, between the front and rear points of the guide rails 21, 21′ corresponds to the distance by which the needle unit is lowered from the first position into the second position onto the tissue surface 9 during the pivoting movement. In the embodiment shown, the lever arms 13 and 14, for stabilizing purposes, are connected between the pins 22, 22′ and 23, 23′ by struts 24 and 25.

The parallel displacement is compensated by a displacement of the pins inside the guide rails and, consequently, also by a displacement of the housing rotation points during the pivoting movement. In FIG. 8, the pins 22, 22′ are arranged in a first position above the tissue surface 9 in a front position inside the guide rails 20, 20′, and the pins 23, 23′ are arranged in a lower position in the guide rails 21, 21′. During the pivoting movement of the lever arms 13 and 14 about their housing rotation points on the pins 22, 22′, the pins 23, 23′ slide along the curved inner face of the guide rails 21, 21′ and are guided by this surface into an upper position in the guide rails 21, 21′, as is shown in FIG. 9. The lever arms 13 and 14 are drawn back by the distance of the parallel displacement of the needle unit 7, the pins 22, 22′ being moved in the guide rails 20, 20′ from the front position to a rear position. In this way, the front end 10 of the lever arms 13 and 14 remains in the same parallel position above the tissue surface 9 during the pivoting movement. During their pivoting movement from the first position to the second position, the lever arms 13 and 14 are thus drawn back by the curved profile of the guide rails 21, 21′ in such a way that the needle unit 7 is inserted into the tissue surface 9 along a straight line. In doing so, the lever arms 13 and 14 at the same time pivot about the bearings 18 and 19 on the beam 15 of the receiving element 2, in order to maintain the angle position of the needle unit 7 relative to the tissue surface 9. Overall, a rectilinear guided movement of the needle unit 7 in the direction of the tissue is obtained.

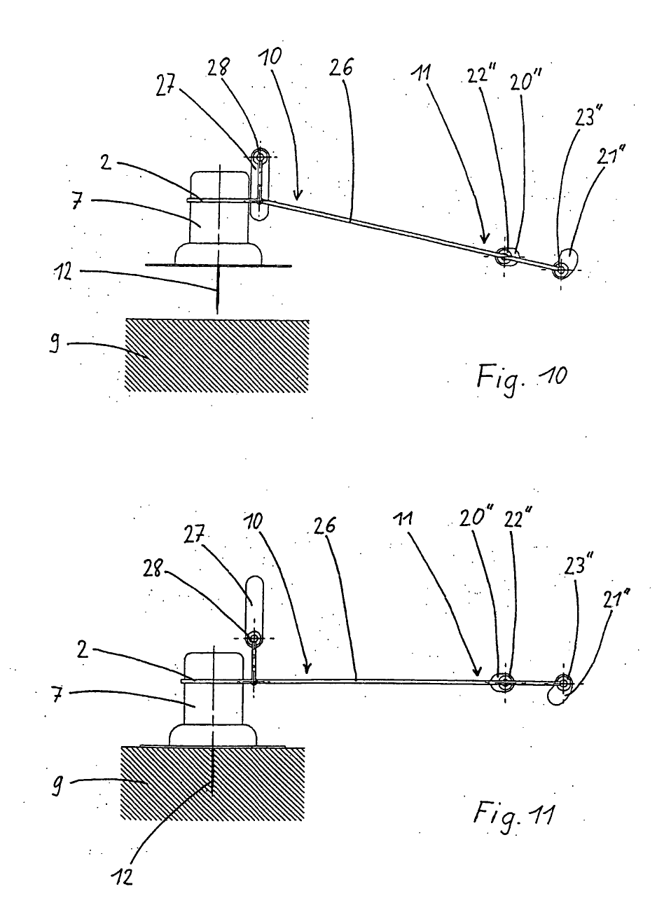

FIGS. 10 and 11 show a fourth embodiment of the insertion device according to the invention in which the support with the receiving element 2 for the needle unit 7 has just one lever arm 26. Secured at the rear end 11 of the lever arm 26 there are pins 22″ and 23″ which each engage in a respective guide rail 20″ and 21″ on the housing of the insertion device. The guide rails 20″ and 21″ and the pins 22″ and 23″ are as described in FIGS. 8 and 9. In the housing there is a further guide rail 27 for a pin 28 arranged at the front end 10 of the lever arm 26. The guide rail 27 extends parallel to a rectilinear movement of the needle unit 7. The pin 28 is arranged on the upper end of a beam 15 extending parallel to the direction of movement of the needle unit 7, and it engages in the guide rail 27.

In FIG. 10, the insertion device is arranged in a first position above the tissue surface 9. The pin 28 is located in an upper position in the guide rail 27, the pin 22′ in a front position in the guide rail 20″, and the pin 23″ in a lower position in the guide rail 21″. During the pivoting movement of the lever arm 26 about its housing rotation point on the pin 22″, the pins 22″ and 23″ move in the guide rails 20″ and 21″, as described in FIGS. 8 and 9, and thus correct the parallel displacement of the front end 10 of the lever arm 26 relative to the tissue surface 9. During the pivoting movement of the lever arm 26, i.e. the rectilinear movement of the needle unit 7, the pin 28 is moved in the guide rail 27 from the upper position to a lower position, as a result of which a second position of the lever arm 26 is obtained with a needle 12 of the needle unit 7 inserted into the tissue surface 9, as is shown in FIG. 11. In this pivoting movement, the lever arm 26 turns relative to the receiving element 2 for the needle unit 7 about a receiver rotation point formed by a bearing of the lever arm 26 on the receiving element 2. By virtue of the guiding of the receiving element 2 via the pin 28 in the guide rail 27 and the turning movement about the receiver rotation point, the needle unit 7 maintains its rotation angle position relative to the tissue surface 9 during the pivoting movement of the lever arm 26. By means of the guide device made up of the guide rails 20, 21 and 27, in this embodiment the change in the rotation angle position caused by the pivoting movement and the parallel displacement relative to the tissue surface 9 in the needle unit are corrected such that the needle unit 7 can be inserted along a straight line into the tissue surface 9.

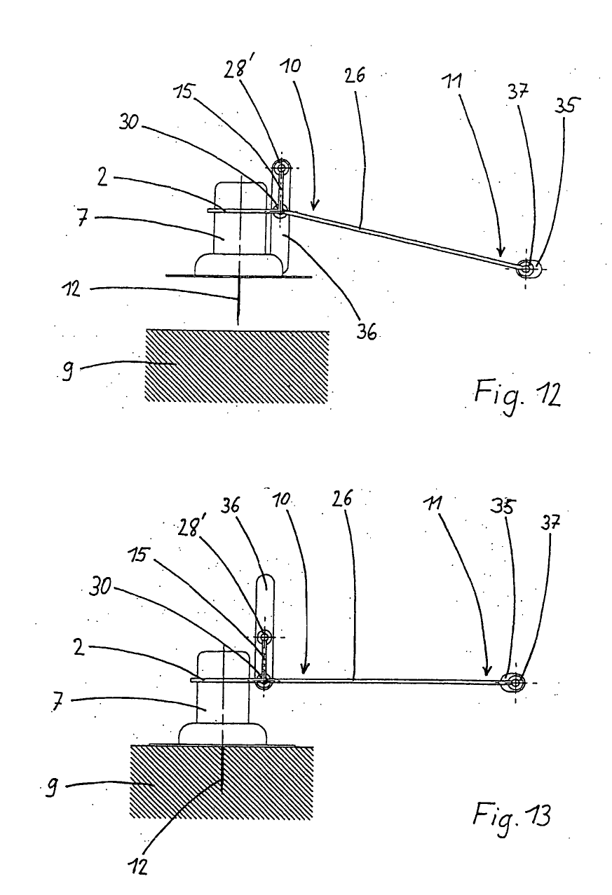

FIGS. 12 and 13 show a fifth embodiment of an insertion device according to the invention in which the guide device comprises a straight guide rail 35 extending perpendicular to the direction of movement of the needle unit 7, with a pin 37 for the rear end 11 of a lever arm 26, and a guide rail 36 extending parallel to the direction of movement of the needle unit 7 for the front end 10 of the lever arm 26. The guide rail 35 corresponds substantially to the guide rails 20, 20′ from FIGS. 8 and 9. In this embodiment, the beam 15 of the receiving element 2 extending parallel to the direction of movement of the needle unit 7 has a pin 28′ arranged at its upper end, while its lower end has a pin 30 which forms the receiver rotation point. In FIG. 12, the insertion device is shown in a first position above the tissue surface 9, with the pin 37 in a front position in the guide rail 35 and with the pins 28 and 30 in an upper position in the guide rail 36. During the pivoting movement of the lever arm 26, the lever arm 26 pivots about the housing rotation point arranged on the pin 37 and, by turning about the receiver rotation point at its front end, is guided along a straight line. As is shown in FIGS. 12 and 13, the pin 37 is moved from a front position to a rear position, and the pins 28 and 30 are moved from an upper position to a lower position, the parallel displacement of the front end of the lever arm 26 relative to the tissue surface 9 being corrected. By virtue of the rectilinear guiding of the pin 28′ and 30 and the turning about the receiver rotation point 29, the rotation angle position of the receiving element 2 for the needle unit 7 relative to the tissue surface 9 is maintained. In this embodiment too, it is accordingly possible to compensate both the change in the rotation angle position and also the parallel displacement of the needle unit 7 during the pivoting movement of the lever arm 26.

In addition to their guiding function, the guide rails can also serve as abutments for the second position with a needle inserted into the tissue, by which means this position can be exactly predetermined. The illustrated embodiments of the guide rails for the guide device of the insertion device are to be understood as examples. Other combinations of guide rails are of course also conceivable.

While exemplary embodiments, including preferred embodiments, of the present invention have been described herein, it is contemplated that various modifications could be made without deviating from the spirit of the present invention. Accordingly, it is intended that the scope of the present invention be dictated by the appended claims.

1. A device for inserting a needle unit at least partially into tissue, comprising:
a housing;
a support with a receiving element which receives the needle unit in a releasable manner, said support being movable relative to the housing, and
an actuation element for triggering the movement of the support,
wherein the support is mounted pivotably about an axis inside the housing.

2. The device as claimed in claim 1, wherein the support can be pivoted from a first position above the tissue to a second position in which the needle unit is inserted at least partially into the tissue.

3. The device as claimed in claim 2, wherein the support comprises at least one lever arm having a front end and a rear end, which, at the rear end, is mounted pivotably inside the housing and, at a front end, has the receiving element.

4. The device as claimed in claim 1, wherein during a pivoting movement of the support, the receiving element maintains its rotation angle position relative to the tissue.

5. The device as claimed in claim 3, wherein the at least one lever arm has, at the rear end, a housing rotation point about which the at least one lever arm turns relative to the housing, and, at the front end, a receiver rotation point about which the receiving element turns relative to the at least one lever arm.

6. The device as claimed in claim 1, wherein the support comprises at least two lever arms which are arranged offset in the direction of pivoting.

7. The device as claimed in claim 6, wherein the at least two lever arms are arranged in parallel alongside one another in the direction of pivoting.

8. The device as claimed in claim 1, further comprising a guide for guiding the support in such a way that the needle unit is guided rectilinearly during the pivoting movement of the support.

9. The device as claimed in claim 8, wherein the guide comprises at least one guide rail on the housing or on the support, into which guide rail there engages at least one pin which is arranged on the support or on the housing.

10. The device as claimed in claim 1, further comprising a guide rail extending substantially perpendicularly to the direction of insertion of the needle unit into the tissue.

11. The device as claimed in claim 1, further comprising a guide rail extending substantially parallel to the direction of insertion of the needle unit.

12. The device as claimed in claim 3, further comprising a guide rail, said guide rail curved in such a way that the front end of the lever arm is guided rectilinearly during a pivoting movement of the lever arm.

13. The device as claimed in claim 2, wherein the actuation element holds the support in the first position in a prestressed state.

14. The device as claimed in claim 13, wherein the actuation element holds the support in a prestressed state by means of a spring.

15. The device as claimed in claim 14, wherein the actuation element comprises a trigger and, when the trigger is actuated, the support is released from the prestressed state and is brought into the second position.

16. The device as claimed in claim 15, wherein the second position is determined by a limit stop for the support on the housing.

17. A device for at least partially inserting a needle unit into a tissue, the device comprising a housing, a support with a receiving element and an actuation device, wherein the needle unit is detachably received by the receiving element and the support can be displaced relative to the housing about an axis from a first position to a second position in which the needle unit is at least partially inserted in the tissue.

18. The device as claimed in claim 17, wherein the support comprises at least one lever arm having two ends and, at one end, a housing rotation point about which the at least one lever arm turns relative to the housing, and, at the other end, a receiver rotation point about which the receiving element turns relative to the at least one lever arm.
